(19) United States
(12) Patent Application Publication
(10) Pub. No.: US 2026/0083404 A1
(43) Pub. Date: Mar. 26, 2026

(54) SIGNAL PROCESSING METHOD OF PREDICTIVE PADDING

(71) Applicant: National Yang Ming Chiao Tung University, Hsinchu City (TW)

(72) Inventors: Chun-Shu WEI, Hsinchu County (TW); Yu-Cheng CHANG, Kaohsiung City (TW); Pin-Hsuan CHAO, Taipei City (TW); Yen-Ming KUAN, Taoyuan City (TW)

(21) Appl. No.: 19/076,112

(22) Filed: Mar. 11, 2025

(30) Foreign Application Priority Data
Sep. 20, 2024 (TW) 113135876

(51) Int. Cl. A61B 5/00; A61B 5/31; G06F 18/10

(52) U.S. Cl. CPC A61B 5/7203; A61B 5/31; G06F 18/10

(57) ABSTRACT

A signal processing method of predictive padding is used to perform predictive padding on an original signal and generate a predictive padding signal accordingly. The signal processing method for predictive padding includes a first kernel function process, a predictive padding process, and a signal merging process. The first kernel function process operates the original signal with a first kernel function to extract a task-related signal and a noise signal. The predictive padding process pads one end of the task-related signal with a predicted extrapolation signal and pads one end of the noise signal with a constant value signal. The signal merging process merges the task-related signal padded with the predicted extrapolation signal and the noise signal padded with the constant value signal to generate the predictive padding signal.

### CROSS REFERENCE TO RELATED APPLICATIONS

This Non-provisional application claims priority under 35 U.S.C. § 119(a) on Patent Application No(s). 113135876 filed in Taiwan, Republic of China on Sep. 20, 2024, the entire contents of which are hereby incorporated by reference.

### BACKGROUND

### Technology Field

This disclosure relates to a signal processing method and, in particular, to a signal processing method of predictive padding.

### Description of Related Art

In general, when performing data operations such as data compression, transmission, or AI (artificial intelligence) model learning, the data padding technology can be used to obtain better data representations. The most commonly used data padding technique is to use the boundary data properties or zero values of the original input signal as the padding value.

Although the above-mentioned padding technology has advantages such as maintaining the size of the output signal and improving the spectrum resolution, it still has some problems. For example, the zero padding can be applied to the boundary data in image processing to avoid the missing edge image. However, the image will still be blurred.

In addition, convolution is a commonly used operation unit in signal processing for data operations such as AI model learning, and it is widely used in applications such as filters and convolutional neural networks. However, convolution operations may often lead to the loss of boundary information of signals, thereby affecting the performance of downstream tasks. In brief, the conventional convolution operations often lose the boundary information of signals, which greatly affects the performance of downstream tasks. In order to solve this problem, a variety of padding methods have been developed, such as the common zero padding, reflection padding and replication padding. Zero padding is to add zeros to the input signal to adjust the data domain of the input signal to meet the input requirements of the calculation model. Reflection padding is to pad the edge pixels of the image by mirror mapping, so as to complete the data boundary. Replication padding can repeatedly pad the data boundary of the image. For example, in the image processing, the pixel value of the data boundary is directly used for padding. However, these conventional padding methods are not designed for the performance of specific downstream tasks. That is, these conventional padding methods cannot effectively improve the performance of downstream tasks.

Furthermore, in the signal processing, zero padding may cause a huge level difference between the original signal and the padding value, which will produce a ringing effect on the original signal after filtering. The ringing effect is a kind of distortion occurred at the boundary of the signal during signal transition. For example, the ringing effect, in images or videos, is also called a ringing artifact, which is manifested as a fuzzy ring or ring artifact at the edge of the original image. These artificial distortions not only affect the clarity of an image, but also lead to a misinterpretation of its content, thereby affecting the performance of downstream tasks. However, the zero padding is often the culprit that exacerbates this phenomenon.

After reviewing various known signal padding techniques, it is found that no signal padding technique has been combined with predictive coding theory. Therefore, it is desired to provide a padding technique that uses task-related future information to effectively improve the performance of downstream tasks.

### SUMMARY

An objective of this disclosure is to provide a signal processing method of predictive padding that can ensure the signal boundaries of task-related information to be clearer and smoother, thereby improving the signal processing performance of downstream tasks.

To achieve the above, this disclosure provides a signal processing method of predictive padding, which is used to perform predictive padding on an original signal and generate a predictive padding signal accordingly. The signal processing method includes a first kernel function process, a predictive padding process and a signal merging process. The first kernel function process operates the original signal with a first kernel function to extract a task-related signal and a noise signal. The predictive padding process pads one end of the task-related signal with a predicted extrapolation signal and pads one end of the noise signal with a constant value signal. The signal merging process merges the task-related signal padded with the predicted extrapolation signal and the noise signal padded with the constant value signal to generate the predictive padding signal.

In one embodiment, the signal processing method of predictive padding further includes a second kernel function process for operating the predictive padding signal with a second kernel function to generate a downstream task input signal.

In one embodiment, the predicted extrapolation signal is padded on two ends of the task-related signal, and the constant value signal is padded on two ends of the noise signal.

In one embodiment, the constant value signal is an average value of the noise signal.

In one embodiment, the constant value signal is a padding signal for reducing an amplitude of the noise signal.

In one embodiment, the predicted extrapolation signal is a predicted future signal output based on the first kernel function.

In one embodiment, the original signal is an electric wave signal

In one embodiment, the original signal is an electroencephalography (EEG) signal.

### BRIEF DESCRIPTION OF THE DRAWINGS

The disclosure will become more fully understood from the detailed description and accompanying drawings, which are given for illustration only, and thus are not limitative of the present disclosure, and wherein:

FIG. 1 is a schematic diagram showing the change of the original signal when processing the original signal with the conventional signal processing method of zero padding;

FIG. 2A is a flow chart of a signal processing method of predictive padding according to an embodiment of this disclosure;

FIG. 2B is a schematic diagram showing the change of the original signal when processing the original signal with the signal processing method of predictive padding of FIG. 2A;

FIG. 3A is a flow chart of a signal processing method of predictive padding according to another embodiment of this disclosure;

FIG. 3B is a schematic diagram showing the change of the original signal when processing the original signal with the signal processing method of predictive padding of FIG. 3A; and

FIG. 4 is a schematic diagram showing the comparison of the downstream task input signal processed by the signal processing method of predictive padding of this disclosure and the downstream task input signal processed by the conventional zero padding.

### DETAILED DESCRIPTION OF THE DISCLOSURE

The present disclosure will be apparent from the following detailed description, which proceeds with reference to the accompanying drawings, wherein the same references relate to the same elements.

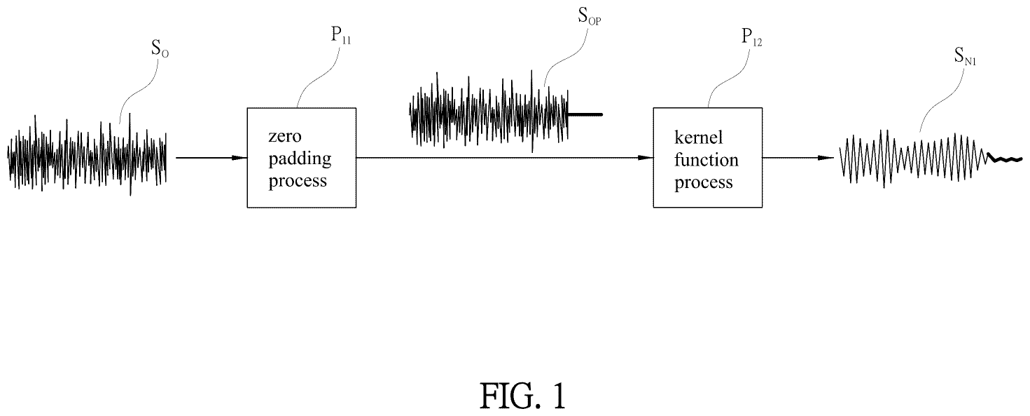

FIG. 1 is a schematic diagram showing the change of the original signal when processing the original signal with the conventional signal processing method of zero padding. As shown in FIG. 1, the conventional signal processing method of zero padding includes a zero padding process P₁₁ and a kernel function process P₁₂. The zero padding process P₁₁ is to add zero to at least one end of the original signal S_{O} so as to generate a zero padding signal S_{OP}. Then, the kernel function process P₁₂ provides a kernel function to process the zero padding signal S_{OP}, thereby generating a downstream task input signal S_{N1}.

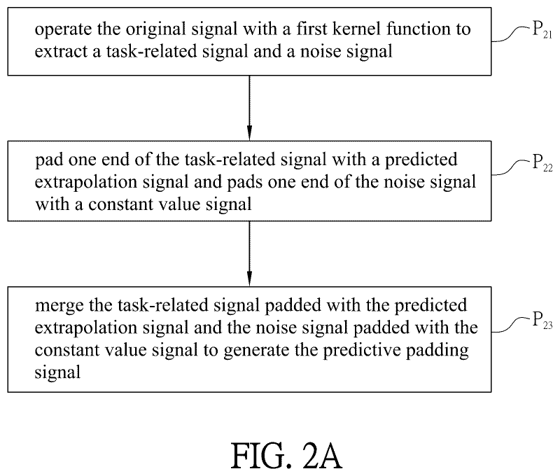

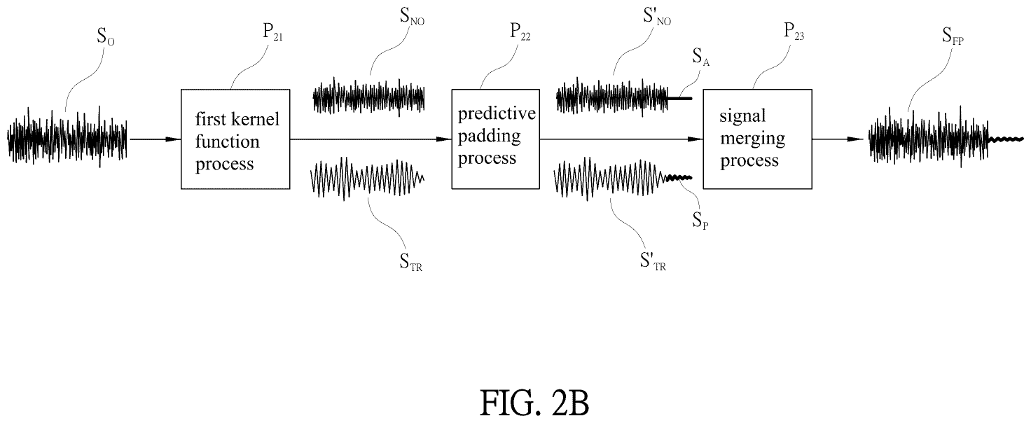

Referring to FIGS. 2A and 2B, the signal processing method of predictive padding according to an embodiment of this disclosure includes a first kernel function process P₂₁, a predictive padding process P₂₂, and a signal merging process P₂₃.

The first kernel function process P₂₁ operates the original signal S_{O} with a first kernel function to extract a task-related signal S_{TR} and a noise signal S_{NO}.

The predictive padding process P₂₂ pads one end of the task-related signal S_{TR} with a predicted extrapolation signal S_{P} and pads one end of the noise signal S_{NO} with a constant value signal S_{A}. To be noted, in response to the requirements of downstream tasks, the predicted extrapolation signal S_{P} or the constant value signal S_{A} can be padded at both ends of the task-related signal S_{TR} or the noise signal S_{NO}. In this embodiment, the predicted extrapolation signal S_{P} can be generated by extrapolating the signal feature of the task-related signal S_{TR} extracted by the first kernel function. Therefore, the predicted extrapolation signal S_{P} can also be interpreted as the predicted future signal outputted by the first kernel function. The constant value signal S_{A} may be the average value of the noise signal S_{NO}, which may be called the baseline level or DC offset. To be noted, in the present disclosure, the padding value of the noise signal S_{NO} may include any signal that assists to reduce the noise amplitude, such as, for example but not limited to, the average value of the noise signal S_{NO}.

The signal merging process P₂₃ merges the task-related signal S′_{TR}, which is padded with the predicted extrapolation signal S_{P}, and the noise signal S′_{NO}, which is padded with the constant value signal S_{A}, to generate a predictive padding signal S_{FP}.

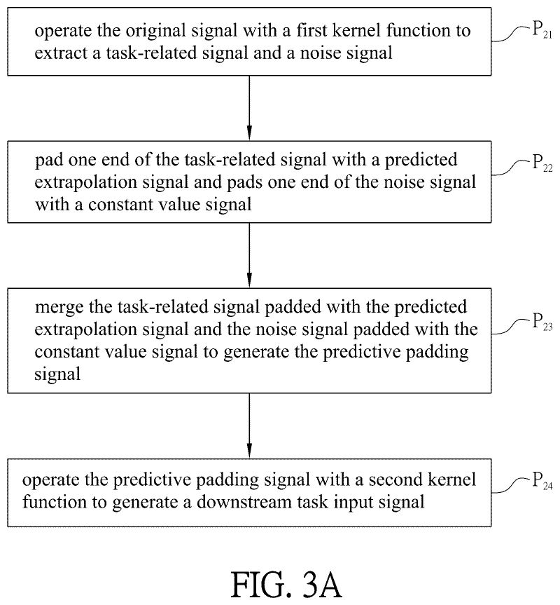

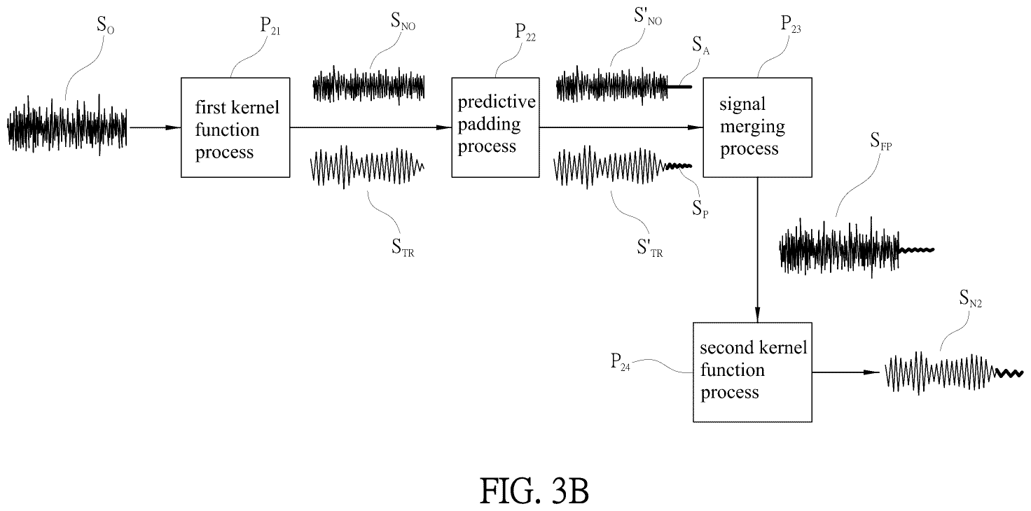

Referring to FIGS. 3A and 3B, the signal processing method of predictive padding of this disclosure may further include a second kernel function process P₂₄ for operating the predictive padding signal S_{FP} with a second kernel function to generate a downstream task input signal S_{N2}.

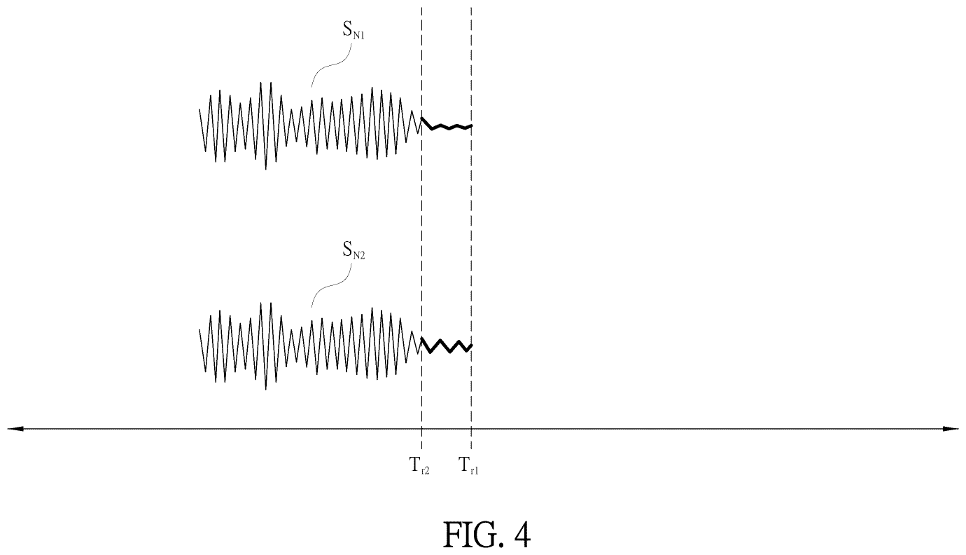

As shown in FIG. 4, the signal processing method of predictive padding of the present disclosure can divide the original signal into multiple signals (e.g. a task-related signal and a noise signal), and then perform targeted predictive padding and average value padding on the boundary information of the extracted signals for generating the downstream task input signal S_{N2}. Thus, compared with the downstream task input signal S_{N1} generated by the signal processing method of conventional zero padding, it is obvious that a better downstream task input signal S_{N2} can be generated. More specifically, the signal in the period from Tᵣ₁ to Tᵣ₂ is more useful, because as shown in FIG. 4, the signal processing method of predictive padding of this disclosure can process the noise signal S_{NO} with average padding so as to effectively suppressing the noise interference, and use the predicted extrapolation signal S_{P} to make the signal boundary of the downstream task input signal S_{N2} more obvious and smoother. Therefore, when the downstream task input signal S_{N2} is used as the input signal of the downstream task analysis processing, the analysis result of the downstream task analysis processing can be made clearer and better.

Reference to FIG. 4, the signal boundary of the downstream task input signal S_{N2} generated by the signal processing method of predictive padding of the present disclosure is less susceptible to noise interference and has relevant information about future prediction. Thus, the generated downstream task input signal S_{N2} can be utilized in many applications. For example, the applicable industries may include finance, medical, manufacturing, communications or software. The applicable products may include financial analysis software, medical equipment software, image processing software, audio processing software, or communication systems. The financial analysis software can, for example, predict currency fluctuations based on national policies. The medical equipment software can, for example, predict the possibility of disease occurrence based on the patient's physiological signals. The image processing software can, for example, restore clearer original images based on grayscale differences. The audio processing software can, for example, restore smoother original sounds based on audio differences. The communication systems can, for example, use the garbled signal generated between smart home appliances to restore and simulate the original commands issued by the user.

To be noted, in this disclosure, the original signal may be an electric wave signal. In the medical industry, the original signal may be an electroencephalography (EEG) signal. In addition, as shown in FIG. 4, since the signal boundary (signal between Tᵣ₁ and Tᵣ₂) of the downstream task input signal S_{N2} generated by the signal processing method of predictive padding of the present disclosure is smoother and clearer, it is relatively easy to measure the phase angle of the signal between Tᵣ₁ and Tᵣ₂ at a certain time point, or it is effective to use the signal between Tᵣ₁ and Tᵣ₂ for prediction.

In summary, the signal processing method of predictive padding of this disclosure includes the first kernel function process P₂₁ for operating the original signal S_{O} to extract a task-related signal S_{TR} and a noise signal S_{NO}, the predictive padding process P₂₂ for padding the task-related signal S_{TR} with a predicted extrapolation signal S_{P} and padding the noise signal S_{NO} with a constant value signal S_{A}, and the signal merging process P₂₃ for generating the predictive padding signal S_{FP}. The signal processing method further includes the second kernel function process P₂₄ for operating the predictive padding signal S_{FP} to generate a downstream task input signal S_{N2}. Accordingly, the signal processing method of predictive padding of this disclosure can ensure the signal boundaries of task-related information to be clearer and smoother, thereby improving the signal processing performance of downstream tasks.

Although the disclosure has been described with reference to specific embodiments, this description is not meant to be construed in a limiting sense. Various modifications of the disclosed embodiments, as well as alternative embodiments, will be apparent to persons skilled in the art. It is, therefore, contemplated that the appended claims will cover all modifications that fall within the true scope of the disclosure.

What is claimed is:

1. A signal processing method of predictive padding, which is used to perform predictive padding on an original signal and generate a predictive padding signal accordingly, comprising:
a first kernel function process operating the original signal with a first kernel function to extract a task-related signal and a noise signal;
a predictive padding process padding one end of the task-related signal with a predicted extrapolation signal and padding one end of the noise signal with a constant value signal; and
a signal merging process merging the task-related signal padded with the predicted extrapolation signal and the noise signal padded with the constant value signal to generate the predictive padding signal.

2. The signal processing method of predictive padding of claim 1, further comprising:
a second kernel function process operating the predictive padding signal with a second kernel function to generate a downstream task input signal.

3. The signal processing method of predictive padding of claim 1, wherein the predicted extrapolation signal is padded on two ends of the task-related signal, and the constant value signal is padded on two ends of the noise signal.

4. The signal processing method of predictive padding of claim 1, wherein the constant value signal is an average value of the noise signal.

5. The signal processing method of predictive padding of claim 1, wherein the constant value signal is a padding signal for reducing an amplitude of the noise signal.

6. The signal processing method of predictive padding of claim 1, wherein the predicted extrapolation signal is a predicted future signal output based on the first kernel function.

7. The signal processing method of predictive padding of claim 1, wherein the original signal is an electric wave signal.

8. The signal processing method of predictive padding of claim 7, wherein the original signal is an electroencephalography (EEG) signal.
